(19) United States
(12) Patent Application Publication
(10) Pub. No.: US 2002/0045242 A1
(43) Pub. Date: Apr. 18, 2002

(54) Culture medium for culturing Lactobacillus clearans, and method for preserving said strain

(76) Inventors: Tadayo Hata, Tondabayashi-shi (JP); Toshiyuki Maruoka, Toyonaka-shi (JP)

Correspondence Address: ARMSTRONG,WESTERMAN, HATTORI,, MCLELAND & NAUGHTON, LLP, 1725 K STREET, NW, SUITE 1000, WASHINGTON, DC, 20006, US

(21) Appl. No.: 09/490,050

(22) Filed: Jan. 24, 2000

(30) Foreign Application Priority Data
Jan. 25, 1999 (JP) H11(1999)-015177

(51) Int. Cl. C12N 1/20; C12N 1/04

(52) U.S. Cl. 435/252.9; 435/253.6; 435/260

(57) ABSTRACT

The culture and preservation of Lactobacillus clearans require special considerations because the titer readily decreases. There is thus an urgent need for culture media and preservatives to prevent such a decrease in bacterial titer during both subculture and storage. The present invention relates to a culture medium in which at least one or more of sodium sulfide and ammonia is decreased by Lactobacillus clearans during culture with the addition of at least one or more of sodium sulfide and aqueous ammonia, as well as to a method for preserving Lactobacillus clearans in which at least one or more of a sulfur-containing amino acid, ovalbumin, bile powder, trehalose, raffinose, dead yeast cells, chlorella, rice bran, bran, soybean milk, and carrot juice is or are present as a preservative around the bacteria.

### BACKGROUND OF THE INVENTION

1. Field of the Invention

The present invention relates to a culture medium that is suitable for culturing Lactobacillus clearans, which has been isolated and selected by the inventors, and to a method for preserving Lactobacillus clearans.

2. Description of the Related Art

The cycle of the natural world is one of creation and decay, from which no biological substance is spared. Swamps, too, overflowing with slime born of the decay of proteins released into the natural world, eventually become purified, allowing the earth to remain beautiful in its own fashion since time immemorial. The mechanisms by which this occurs have long remained unknown, but are now known to primarily involve the behavior of microorganisms, leading to the contemporary development of various purification systems, including activated sludge processes, and their application in treatment plants for life wastewater and plant wastewater. Given the complexity concerning the question of what sort of properties characterize the bacteria involved in such purification, it is no wonder that further specialized analysis and corroboration are needed.

A broad and diverse array of substances are the object of such purification, and it would ultimately be impossible to test all of them. Our attention was accordingly focused on odoriferous substances (most of which are products of putrefaction and are noxious) which are readily and rapidly determinable by smell, and are broadly classified into odoriferous sulfur compounds, odoriferous nitrogen compounds, and odoriferous carbon compounds. After considerable research, studies on low molecular odoriferous compounds, that is, odoriferous sulfur compounds such as sodium sulfide and methyl sulfide, odoriferous nitrogen compounds such as ammonia, indole, and skatole, and odoriferous carbon compounds such as acetic acid and butyric acid, have shown conclusively that the objectives were sufficiently achieved, allowing dramatic progress to be made in research on purification and deodorization. That is, clearance-bacteria capture these odoriferous substances as food, not as poison, and use them as cell components for themselves or as an energy source, while bacteria having particularly potent action have a deodorizing capacity. A great number of bacteria having such characteristics are found throughout the natural world, and toil around the clock in the work of purification to which they are most suited. Our very bodies are a microcosm, the enteric canal in particular being an organ directly linked to the external world as part of the natural environment itself. There should be no wonder, therefore, that “experts” in purification exist in the intestines.

Based on the above findings, the inventors studied enteric clearance-bacteria among the nonpathogenic bacteria capable of living in the intestines. That is, the inventors singled out the Lactobacillus genus, which occurs widely in the natural world, from within the living body such as in the enteric canal, the mouth, and the vagina, to grasses, tree leaves, agricultural fruits, fermented food products, soil, and sewage. As a result, they ascertained the existence of a previously unknown group belonging to the Lactobacillus genus capable of exhibiting a potent purifying capacity in the intestines. These bacteria include a considerably wide range of species currently classified as belonging to the Lactobacillus genus, such as L. casei, L. salivarius, L. brevis, and L. plantarum, and are collectively referred to as Lactobacillus clearans .

Lactobacillus clearans, which are novel lactobacilli capable of decreasing sodium sulfide and ammonia (Japanese Examined Patent Application (Kokoku) 4-632), are useful bacteria that exhibit a potent purifying capacity in the intestines through their capacity to decrease sodium sulfide, ammonium sulfide, methyl mercaptane, ethyl mercaptane, dimethyl sulfide, diethyl sulfide, acetaldehyde, skatole, indole, methylamine, ethylamine, diethylamine, triethylamine, and the like. The inventors thus found these species of the Lactobacillus genus capable of exhibiting a potent purifying capacity in the intestines, discovered as a result of bacteriological research that these species had entirely novel functions, and were awarded a patent for these species (1714431). It has become apparent that Lactobacillus clearans does not merely decrease odoriferous noxious substances in the intestines, but are also a group forming intestinal flora, which synthesize vitamins and amino acids and control the growth of extrinsic bacteria, having a tremendous effect on groups such as bacteria which may be considered beneficial bacteria having action that is good for the living body such as immunoactivating action, typically groups belonging to the Lactobacillus genus and Bifidobacterium genus, and bacteria which conversely may be considered deleterious because of their noxious and pathogenic nature, typically groups belonging to Veillonella and Clostridium, such as Welchii, and furthermore control the growth of pathogenic bacteria, reduce their toxicity, and so forth. Table 1 shows the functional differences between Lactobacillus clearans and conventionally known species of the Lactobacillus genus.

TABLE 1
Comparison of functions between Lactobacillus clearans
and conventional species of the Lactobacillus genus

| | | Conventional species of |
|---|---|---|
| Parameter | Lactobacilius clearans | Lactobacillus |
| Vs. enteral putrefying, | Decreases most | Utilizes and degrades |
| odoriferous, noxious | odoriferous noxious | odoriferous carbon |
| substances-sulfur | compounds-sulfur | compounds, but has no |
| compounds, nitrogen | compounds, nitrogen | action on odoriferous |
| compounds, and carbon | compounds, and carbon | noxious compounds such |
| compounds | compounds-by | as sulfur compounds and |
| | utilizing, degrading, | nitrogen compounds |
| | and denaturing them | |
| Fecal deodorization | ++ | − to ± |
| Action on beneficial | Causes considerable | Grow when individuals |
| bacteria commonly | growth | continue to ingest |
| present in intestines | | |
| Bifidobacterium | 2 to 10 times | 1 to 3 times |
| Lactobacillus | 10 to 100 times | 10 times < |
| Action on deleterious | Strongly suppressed by | Has suppressing action. |
| bacteria commonly | considerable growth of | but not much can be |
| present in intestines | beneficial bacteria | anticipated |
| Veillonella | 1/20 to 1/100 | 1 to 1/5 |
| Clostridium | 1/20 to 1/100 | 1 to 1/5 |
| Anti-flatulent action | ++ | − to + |
| Nutritional requirement | low to moderate | high nutrition |
| Intestinal growing | + | − to + |
| ability | | |
| Intestinal stationary | − to + | − |
| ability | | |
| Action on pathogens | Rendered non-pathogenic | No effect |
| during symbiosis | (S-R conversion) | |
| Salmonella | Pathogenicity | Eradicated in struggle |
| | inactivated by | with pathogens over |
| | 47^{th} co-subculture | several generations of |
| | | subculture with all |
| Shigella | Pathogenicity | pathogens |
| | inactivated by | |
| | 108^{th} co-subculture | |
| E. coli (0-157) | Pathogenicity | |
| | inactivated by | |
| | 18^{th} co-subculture | |

When considering why Lactobacillus clearans was able to be produced, the following can be inferred. That is, the molten earth born 4,600,000,000 years ago cooled, producing water vapor, which formed the first seas. As yet, there was still no oxygen, however, and the sea was composed of acidic hot water containing sulfur, iron, and the like produced by magma. Organic substances such as amino acids and nucleic acids were synthesized a little at a time by chemical reactions in the sea, which aggregated and condensed in the form of oily drops. Eventually, life forms were born of these oily drops and continued to grow, consuming organic materials accumulating in the sea to the verge of extinction. However, from among these life forms there appeared anaerobic bacteria capable of using inorganic materials such as the sulfur dissolved in the sea to acquire energy and of synthesizing organic materials from carbon dioxide. These anaerobic bacteria evolved over long periods of time, differentiating into the first photosynthetic bacteria which used energy to release oxygen, and the ancient predecessors of the Lactobacillus genus occurring today. These played an active role in the purification of odoriferous noxious substances from magma, thanks to which most noxious substances were precipitated to the ocean floor, while oxygen also increased, allowing an environment hospitable to life to continue to gradually flourish and form the building blocks for the subsequent explosive phenomenon of life. At sometime during this process, most Lactobacillus succumbed in the struggle for growth with other bacteria which had become acclimated to, and had adapted to, the harsh environment of the immense natural world, escaping to dwell in nutrient-rich areas replete with the existence of carbohydrates, amino acids, vitamins, and the like, and to areas with a milder, more constant environment, after which their inherent purifying power, that is, the purifying power against odoriferous and noxious sulfur compounds and the purifying power against odoriferous and noxious nitrogen compounds, was gradually lost. The Lactobacillus genus, however, has retained the power to utilize odoriferous carbon compounds to this very day.

Although various well known methods such as lyophilization, ultracold preservation, or liquid, wet, semi-dry, and dry methods or the like can be used as methods for preserving Lactobacillus clearans, it is most important to prevent the loss of the characteristic ability of Lactobacillus clearans to decrease odoriferous and noxious substances during storage, and the next most important issue is to ensure longer viability while preserving this ability. Research by the inventors clearly revealed the need for special consideration to that end. That is, given the presence of substances causing the bacterial titer to rapidly decline during bacterial subculture, not only will the titer gradually decline, but its very survival will be threatened, no matter what method of preservation is used.

Lyophilization is presently the predominant method of preserving bacteria, and Lactobacillus is no exception. Although lyophilization has been used for all products which need to be stored for long periods of times such as anti-flatulents or yogurt strains, the preservability of Lactobacillus is basically not considered to be very good. In fact, attempts at collecting and reviving lyophilized cells of Lactobacillus commercially available domestically and abroad failed to achieve the viable cell count indicated in virtually all products, regardless of the expiration date, among which there were some products in which no viable cells were to be found. This was the case, despite the expertise and the results of research by the manufacturers. Studies by the inventors on Lactobacillus clearans revealed that not only did the viable cell count decline at an early stage in the presence of commonly used preservatives, they even led to a decline in titer.

Lactobacillus clearans may be assumed to be the descendants, or so-called atavistic mutant strains, which have survived to this very day upon the continuous inheritance of purifying action, which their ancestors kept at all costs, against odoriferous sulfur compounds, nitrogen compounds, and carbon compounds. It is impossible to predict what fate will befall the delicate Lactobacillus clearans, which is now in a state of flux between ancient and contemporary Lactobacillus, under human care. Different concerns are mandated for the culture media used to culture such bacteria and the preservatives used to preserve them. In fact, in tests on these bacteria, the titer decreased during both subculture and storage. As such, the paramount issues are what conditions of subculture would allow the potent titer to remain unaffected, and what conditions of storage would allow the potent titer to remain unaffected.

### SUMMARY OF THE INVENTION

As a result of extensive research to remedy these problems, the inventors developed a culture medium capable of sustaining the titer of Lactobacillus clearans in decreasing sodium sulfide and ammonia, and a method of preservation. That is, the present invention is a culture medium for culturing Lactobacillus clearans, comprising the addition of at least one or more of sodium sulfide and aqueous ammonia, so that, during the culture of the Lactobacillus clearans, at least one or more of sodium sulfide and ammonia is decreased by Lactobacillus clearans by the 24th hour of culture, and is a culture medium preferably comprising the addition of sodium sulfide in a concentration of 500 ppm, wherein the sodium sulfide is decreased 10% or more by the 24th hour of culture during the culture of Lactobacillus clearans, and preferably comprising the addition of aqueous ammonia in a concentration of 500 ppm, wherein the ammonia is decreased 10% or more by the 24th hour of culture during the culture of Lactobacillus clearans. The culture medium preferably comprises the addition of at least one or more of odoriferous sulfur compounds, odoriferous nitrogen compounds, and odoriferous carbon compounds, wherein the odoriferous sulfur compound is preferably at least one or more of sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, and derivatives thereof, the odoriferous nitrogen compound is preferably at least one or more of ammonia, skatole, indole, acetanilide, methylamine, dimethylamine, diethylamine, triethylamine, and derivatives thereof, and the odoriferous carbon compound is preferably at least one or more of formic acid, acetic acid, propionic acid, butyric acid, formaldehyde, acetaldehyde, propionaldehyde, crotonaldehyde, phenol, butyl alcohol, amyl alcohol, and derivatives thereof. The culture medium also preferably comprises the addition of at least one or more of sulfur-containing amino acid, glutamic acid, lysine, and aspartic acid as an amino acid, preferably the addition of at least one or more of Vitamin C, Vitamin E, Vitamin B₁₂, calcium pantothenate, folic acid, and nicotinamide as a vitamin, preferably the addition of at least one or more of manganese, zinc, magnesium, and molybdenum as a mineral component, and preferably the addition of at least one or more of chlorella CGF, soybean milk and bile powder.

The second of the present inventions is a method for preserving Lactobacillus clearans, comprising the presence of at least one or more of sulfur-containing amino acid, ovalbumin, bile powder, trehalose, raffinose, dead yeast cells, chlorella, rice bran, bran, soybean milk, and carrot juice as a preservative around the Lactobacillus clearans during the preservation of Lactobacillus clearans, and preferably comprises, in addition to the aforementioned preservative, the addition of at least one or more of glutamic acid, lysine, aspartic acid, Vitamin C, Vitamin E, Vitamin B₁₂, calcium pantothenate, folic acid, nicotinamide, manganese, zinc, magnesium, molybdenum, sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, ammonia, skatole, indole, acetanilide, methylamine, dimethylamine, diethylamine, triethylamine, formic acid, acetic acid, propionic acid, butyric acid, formaldehyde, acetaldehyde, propionaldehyde, crotonaldehyde, phenol, butyl alcohol, amyl alcohol, and derivatives thereof around the Lactobacillus clearans, and furthermore preferably comprises the presence of at least one or more of animal-derived powdered skim milk, ovalbumin, lactose, liver extract powder, and serum, as well as at least one or more of vegetable-derived soybean whey, trehalose, raffinose, starch, chlorella, chlorella CGF, rice bran, bran, alfalfa juice, clover juice, wheat germ extract, soybean milk, tomato juice, carrot juice, grape juice, aloe powder, green tea powder, and dead yeast cells as a preservative, and in addition to the aforementioned preservatives, the addition of at least one or more of glutamic acid, lysine, aspartic acid, Vitamin C, Vitamin E, Vitamin B₁₂, calcium pantothenate, folic acid, nicotinamide, manganese, zinc, magnesium, molybdenum, sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, ammonia, skatole, acetanilide, methylamine, dimethylamine, diethylamine, triethylamine, formic acid, acetic acid, propionic acid, butyric acid, formaldehyde, acetaldehyde, propionaldehyde, crotonaldehyde, phenol, butyl alcohol, amyl alcohol, and derivatives thereof around the Lactobacillus clearans. Additionally, the method for preserving Lactobacillus clearans may comprise any of lyophilization, ultracold preservation, or liquid, wet, semi-dry, or dry methods.

### BRIEF DESCRIPTION OF THE DRAWINGS

FIG. 1 is an illustration of culture with the culture medium for Lactobacillus clearans and the titer assay therein.

### DESCRIPTION OF THE PREFERRED EMBODIMENTS

The Lactobacillus clearans referred to in the present invention are novel strains of the Lactobacillus genus, which have the following biochemical characteristics (1), (2), (3), and (4). To wit, they are strains of Lactobacillus: (1) which can decrease both Na₂S•9H₂O and NH₄OH when 0.5 g Na₂S•9H₂O and/or 0.5 mL NH₄OH is or are added to 5 g meat extract, 5 g peptone, 1 g glucose, 1 g CaCO₃, and 1 L water (neutral pH); (2) which show no growth-promoting action even when 0.5 g Na₂S•9H₂O and/or 0.5 mL NH₄OH is or are added during the logarithmic growth phase during culture of the bacteria in medium comprising 1 g casamino acid and vitamins (A: 900 IU; B₁: 1 mg; B₂: 1 mg; B6: 1 mg; B12: 5 γ; nicotinamide: 16 mg; calcium pantothenate: 8 mg; C: 64 mg; D2: 120 IU) in Stephanson-Whetham medium (abbreviated as S-W; 1 g KH₂PO₄, 0.7 g MgSO₄•7H₂O; 1 g NaCl; 4 g (NH₄)₂HPO₄; 0.03 g FeSO₄•H₂O; 5 g glucose); (3) natural isolated strains show stronger resistance than conventionally known lactobacilli and weaker resistance than Lactobacillus clearans against Na₂S•9H₂O; and (4) which are gram-positive, rods, non-motile, catalase-negative, with no reduction of nitrates, no decomposition of gelatin, no formation of indole or hydrogen sulfide, and high ability to form lactic acid from glucose and lactose, as well as accelerated growth with the addition of acetic acid (Japanese Examined Patent Application (Kokoku) 4-632).

The various media given in Tables 2, 3, 4, 5, and 6, such as the various types of media classified into low nutrient medium, moderate nutrient medium, and high nutrient medium, can be used for the subculture of Lactobacillus clearans.

TABLE 2
Subculture medium composition (1) (composition in 1 L)
Stephanson-Wetham medium

| | |
|---|---|
| KH₂PO₄ | 1 g |
| MgSO₄.7H₂O | 0.7 g |
| NaCl | 1 g |
| (NH₄)₂HPO₄ | 4 g |
| FeSO₄.7H₂O | 0.03 g |
| Glucose | 5 g |

TABLE 3
Subculture medium composition (2) (composition in 1 L)
MRS medium

| | |
|---|---|
| Meat extract | 10 g |
| Yeast extract | 5 g |
| Peptone | 10 g |
| MgSO₄.7H₂O | 0.2 g |
| MnSO₄.5H₂O | 0.5 g |
| Sodium acetate | 5 g |
| Ammonium citrate | 2 g |
| KH₂PO₄ | 2 g |
| Glucose | 20 g |

TABLE 4
Subculture medium composition (3) (composition in 1 L)
Low nutrient medium

| Medium | Composition |
|---|---|
| a-1 | 1 g casamino acid |
| | added to Stephanson-Wetham medium |
| a-2 | 1 g yeast extract |
| | added to Stephanson-Wetham medium |
| a-3 | 1 g casamino acid |
| | 0.1 g vitamin¹⁾ |
| | added to Stephanson-Wetham medium |

¹⁾includes Vitamin A: 900 IU; Vitamin B₁: 1 mg; Vitamin B₂: 1 mg, Vitamin B₁₂: 5 μg; nicotinamide: 16 mg; calcium pantothenate: 8 mg; Vitamin C: 16 mg; Vitamin D₂: 120 IU in 1 g.

TABLE 5
Subculture medium composition (4) (composition in 1 L)
Moderate nutrient medium

| Medium | Composition |
|---|---|
| b-1 | 1 g casamino acid |
| | 0.1 g vitamin¹⁾ |
| | 5 g skim milk |
| | added to Stephanson-Wetham medium |
| b-2 | 5 g meat extract |
| | 5 g peptone |
| | 5 g Glucose |
| b-3 | 1 g MRS medium diluted ½ |

¹⁾includes Vitamin A: 900 IU; Vitamin B₁: 1 mg; Vitamin B₂: 1 mg, Vitamin B₁₂: 5 μg; nicotinamide: 16 mg; calcium pantothenate: 8 mg; Vitamin C: 16 mg; Vitamin D₂: 120 IU in 1 g.

TABLE 6
Subculture medium composition (5) (composition in 1 L)
High nutrient medium

| Medium | Composition |
|---|---|
| c-1 | MRS medium |
| c-2 | 100 g skim milk |
| c-3 | 30 g skim milk |
| | added to MRS medium |

The method for assaying the titer of Lactobacillus clearans is described below; the following are examples of the function which these bacteria have: (1) the capacity for decreasing odoriferous noxious substances such as the sulfur compound sodium sulfide and the nitrogen compound ammonia; (2) the capacity for improving the intestinal flora, that is, the capacity for increasing beneficial bacteria such as Bifidobacterium and Lactobacillus, and for markedly decreasing deleterious bacteria such as Veillonella and Clostridium, including Welchii; and (3) the capacity for suppressing the growth and toxicity of intestinal infectious pathogenic bacteria. Although these three capacities may be individually assayed and comprehensively evaluated, the results of extensive study have revealed that the aforementioned capacities of Lactobacillus clearans are intimately inter-related. As such, the assay of the titer was limited to (1) the capacity for decreasing odoriferous noxious substances in the intestine, which is easily tested and provides rapid results.

The titer assay medium for assaying the capacity for decreasing odoriferous noxious substances in the intestine consisted of 0.5 g sodium sulfide or 0.5 mL aqueous ammonia added to medium comprising 5 g meat extract, 5 g peptone, 5 g glucose, 3 g sodium butyrate, and 3 g calcium carbonate. The medium was inoculated with the test bacterium for anaerobic culture at 37° C., and the decrease in the added sodium sulfide or ammonia over time was determined. At such times, the sodium sulfide was measured by the lead acetate method or the iodine titration method in JIS K 0102-1985, while the ammonia was measured by Nestler's method or the indophenol blue absorbance method in JIS K 0102-1985. The Lactobacillus clearans being assayed is quite diverse; Table 7 gives the percentage of decrease in sodium sulfide and ammonia determined when the titer of three typical strains, namely BHPH-L-1 (FERM P-17149, FERM BP-6971), BHPH-L-2 (FERM P-17148, FERM BP-6972), and BHPH-L-3 (FERM P-17150, FERM BP-6973), was high.

TABLE 7
Percentage of decrease in sodium sulfide and ammonia by
Lactobacillus clearans with high titer

| | Decrease (%) of | | | Decrease (%) | | |
|---|---|---|---|---|---|---|
| | sodium sulfide | | | of ammonia | | |
| FERM No. | 24 hr | 48 hr | 72 hr | 24 hr | 48 hr | 72 hr |
| P-17148 | 20 | 40 | 50 | 15 | 25 | 40 |
| BP-6972 | | | | | | |
| P-17149 | 30 | 50 | 55 | 25 | 40 | 50 |
| BP-6971 | | | | | | |
| P-17150 | 40 | 65 | 70 | 30 | 45 | 65 |
| BP-6973 | | | | | | |

The base culture medium ideally has excellent growth and minimal decrease in titer. This is the most important point related to the yield of bacteria from the practical stand point of mass culture (scaling up). Upon seeking an ideal culture medium, we have successfully found the key. That is, as indicated in Table 8, it became apparent that when culture was managed with the addition of sodium sulfide or ammonia to the base medium, it was essential for the two added substances to be decreased to some extent by the 24 hour, with subcultures in media without any such decrease resulting in a considerable loss of titer, making them ultimately unusable. That is, Table 8 shows that, in cases where 0.5 g sodium sulfide and 0.5 mL aqueous ammonia were added to 1 liter media, the odoriferous substances serving as inherent nutrient components, such as odoriferous sulfur, nitrogen, and carbon substances, were not used as nutrient sources in excessively high nutrient media, and that only easily usable nutrient sources were used, resulting in greater amounts of a bacteria, but with the gradual loss and ultimate inactivation of the bacterial characteristics. Here, aqueous ammonia refers to reagent aqueous ammonia, such as aqueous solution containing 25.0 to 27.9 w/v % ammonia.

TABLE 8
Percentage of decrease in sodium sulfide and ammonia in
culture media to which they had been added for Lactobacillus clearans

| | | | | Initial decrease (%) | | | | | |
|---|---|---|---|---|---|---|---|---|---|
| | | | | Odoriferous | | | Odoriferous | | |
| | | | | sulfur | | | nitrogen | | |
| | | | Test | compounds | | | compounds | | |
| | | Substance | bacterium | 24 | 48 | 72 | 24 | 48 | 72 |
| Type | Medium | added | FERM No. | hr | hr | hr | hr | hr | hr |
| Low | a-1 | Sodium sulfide | P-17148, BP-6972 | 10 | 15 | 25 | 10 | 20 | 30 |
| nutrient | | and | P-17149, BP-6971 | 15 | 20 | 35 | 15 | 30 | 40 |
| medium | | Aqueous ammonia | P-17150, BP-6973 | 20 | 30 | 40 | 20 | 35 | 45 |
| | a-2 | Sodium sulfide | P-17148, BP-6972 | 10 | 15 | 25 | 10 | 15 | 20 |
| | | and | P-17149, BP-6971 | 15 | 20 | 30 | 15 | 25 | 40 |
| | | Aqueous ammonia | P-17150, BP-6973 | 25 | 35 | 40 | 20 | 30 | 40 |
| | a-3 | Sodium sulfide | P-17148, BP-6972 | 15 | 25 | 30 | 15 | 25 | 35 |
| | | and | P-17149, BP-6971 | 20 | 30 | 35 | 20 | 30 | 40 |
| | | Aqueous ammonia | P-17150, BP-6973 | 25 | 35 | 10 | 20 | 35 | 45 |
| Moderate | b-1 | Sodium sulfide | P-17148, BP-6972 | 10 | 15 | 25 | 10 | 15 | 20 |
| nutrient | | and | P-17149, BP-6971 | 15 | 20 | 30 | 15 | 20 | 30 |
| medium | | Aqueous ammonia | P-17150, BP-6973 | 20 | 25 | 30 | 15 | 20 | 25 |
| | b-2 | Sodium sulfide | P-17148, BP-6972 | 0 | 3 | 10 | 10 | 0 | 10 |
| | | and | P-17149, BP-6971 | 0 | 5 | 10 | 0 | 5 | 10 |
| | | Aqueous ammonia | P-17150, BP-6973 | 0 | 5 | 15 | 0 | 5 | 10 |
| | b-3 | Sodium sulfide | P-17148, BP-6972 | 5 | 10 | 15 | 5 | 7 | 10 |
| | | and | P-17149, BP-6971 | 5 | 10 | 15 | 5 | 8 | 10 |
| | | Aqueous ammonia | P-17150, BP-6973 | 5 | 7 | 10 | 5 | 7 | 10 |
| High | c-1 | Sodium sulfide | P-17148, BP-6972 | 0 | 2 | 7 | 0 | 3 | 5 |
| nutrient | | and | P-17149, BP-6971 | 0 | 0 | 3 | 0 | 0 | 2 |
| medium | | Aqueous ammonia | P-17150, BP-6973 | 2 | 3 | 5 | 2 | 5 | 7 |
| | c-2 | Sodium sulfide | P-17148, BP-6972 | 0 | 0 | 8 | 0 | 0 | 5 |
| | | and | P-17149, BP-6971 | 0 | 0 | 10 | 0 | 0 | 5 |
| | | Aqueous ammonia | P-17150, BP-6973 | 0 | 0 | 7 | 0 | 0 | 5 |
| | c-3 | Sodium sulfide | P-17148, BP-6972 | 0 | 0 | 5 | 0 | 0 | 5 |
| | | and | P-17149, BP-6971 | 0 | 0 | 5 | 0 | 0 | 5 |
| | | Aqueous ammonia | P-17150, BP-6973 | 0 | 0 | 10 | 0 | 0 | 5 |

The most important amino acids constituting the bacterial cells or enzymes were studied by type for their effects on Lactobacillus clearans. It was revealed that the addition of specific amino acids, namely, sulfur-containing amino acids such as cystine methionine, cysteine, and taurine, well as glutamic acid, lysine, and aspartic acid, were extremely effective when added during subculture, whereas the addition of proline, tyrosine, and the like brought about a rapid decrease in titer. That is, they could be broadly classified into three groups: certain types of amino acids that helped to retain the titer of Lactobacillus clearans, other types that had less effect, and still others that brought about a significant decrease in titer. This successfully solved the contradiction in conventional experiments, specifically, the contradiction that if a nutrient were improved in an effort to facilitate the growth of bacteria, the nutrient would be excellent for the bacteria, yet the distinguishing characteristic of decreasing noxious substances would be lost. Accordingly, it need hardly be pointed out that the aforementioned effective amino acids could be mixed with the aforementioned odoriferous sulfur, nitrogen, and carbon compounds to prevent the titer from decreasing further.

We looked for substances capable of such enhancement or potentiation and conducted extensive- study. As a result, we discovered that Vitamin C, Vitamin E, Vitamin B₁₂, calcium pantothenate, folic acid, nicotinamide, and the like were effective vitamins. It has become clear that these vitamins are deeply involved in the production of enzymes that decrease odoriferous substances such as odoriferous sulfur, nitrogen, and carbon compounds.

It was also discovered that manganese, zinc, magnesium, molybdenum, and the like are effective minerals. It has become clear that these minerals are deeply involved in the activity of enzymes that decrease odoriferous substances such as odoriferous sulfur, nitrogen, and carbon compounds.

This was confirmed by culturing Lactobacillus clearans in media containing the aforementioned effective vitamins and minerals, and subsequently removing the bacteria, and by then simply adding a portion of the resulting culture broth to a liquid containing an odoriferous substance such as an odoriferous sulfur, nitrogen, or carbon compound, resulting in the decrease of such odoriferous substances.

It was also discovered that chlorella CGF, soybean milk, bile powder, and the like were effective substances for maintaining the titer of Lactobacillus clearans.

In addition to study of the culture components, it is also preferable to subculture bacteria in the logarithmic growth phase in subsequent media during subculture or growth in an effort to prevent any decrease in the titer. It is also preferable to avoid heat denaturation of the medium components during the manufacture and sterilization of the medium.

A method of preservation was then studied, resulting in the conclusion that the most important item is to first produce a favorable preservative. Preservatives commonly used at present in lactobacilli, such as lactose, various types of starch, and skim milk, (1) are easy to handle, (2) are inexpensive, (3) can be administered directly into the living body, and also produce no discomfort when taken. For these and other reasons, there is a strong tendency to use them, primarily for human convenience, even though they are both favorable and unsuitable for lactobacilli. At present, they can be taken in the form of enteric capsules, but we have decided to review these circumstances from the point of view of lactobacilli, without assuming priority on the part of humans, as a way of drawing attention to the lactobacilli. Thus, a wide variety of substances were tested as preservatives, from commonly used protein-based substances in bacterial cultures, to composites of various animal and plant substances and saccharides, for a wide variety of bacterial strains, including the three typical strains of Lactobacillus clearans, that is, BHPH-L-1 (FERM P-17149,FERM BP-6971), BHPH-L-2 (FERM P-17148, FERM BP-6972), and BHPH-L-3 (FERM P-17150,FERM BP-6973). The amounts in which preservatives are added varies widely depending on the type of preservative, with a varying range of suitability, and thus cannot be determined as a matter of absolute principle, but generally ranges, in terms of the weight ratio of preservative solids, from 1 to 500 times that of centrifuged bacterial cells.

The starches referred to in the present invention are not limited to any particular starting material, but examples include soluble starch, corn starch, potato starch, and sweet potato starch.

The dead yeast cells referred to in the present invention refer to yeast in a non-viable state. An example is Baker's yeast, which can be killed by 10 minutes of treatment with 100° C. hot water and then dried. After being killed, however, Baker's yeast may be either in a dry or moist state. Yeast extract powder refers to yeast extract which has been dried and made into a powder. The drying method is not particularly limited.

### EXAMPLES

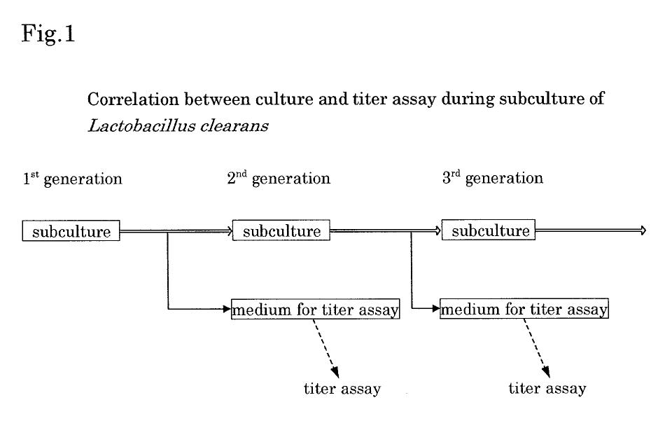

Lactobacillus clearans were subcultured in the various media given in Tables, 2, 3, 4, 5, and 6, media for assaying the titer were inoculated at each stage of the subculture, and the titer was assayed from the first through ninth generations. The procedure is illustrated in FIG. 1, the results of the titer assay are given in Table 9, and Table 10 summarizes all of the strains. The titer is represented by concentric circles to indicate virtually no decrease in titer, by a circle to indicate a slight decrease in, but sufficient retention of, titer, by a triangle to indicate a gradual decrease in titer that was unsuitable for practical purposes, and by an “x” to indicate a considerable loss of titer. Basically, a decrease in titer was noted in all media, increasing in the order from low, to moderate, to high nutrient media. The differences rapidly increased with further subcultures.

TABLE 9
Correlation between number of subcultures and
titer of Lactobacillus clearans in various media

| | | | Decrease | | | | | |
|---|---|---|---|---|---|---|---|---|
| | | | (%) by 72 | | | | | |
| | | | hour in 1^{st} | | Number of subcultures and | | | |
| | | Test bacterium | generation | | titer | | | |
| Type | Medium | FERM No. | S¹⁾ | N²⁾ | 1^{st} | 3^{rd} | 6^{th} | 9^{th} |
| Low | a-1 | P-17148, BP-6972 | 50 | 40 | ⊚ | ⊚ | ◯ | ◯˜Δ |
| nutrient | | P-17149, BP-6971 | 50 | 45 | ⊚ | ⊚ | ◯ | ◯˜Δ |
| medium | | P-17150, BP-6973 | 65 | 60 | ⊚ | ⊚˜◯ | ◯ | Δ |
| | a-2 | P-17148, BP-6972 | 45 | 35 | ⊚ | ⊚ | ◯ | ◯˜Δ |
| | | P-17149, BP-6971 | 45 | 40 | ⊚ | ⊚˜◯ | ◯ | Δ |
| | | P-17150, BP-6973 | 60 | 55 | ⊚ | ⊚˜◯ | ◯ | Δ |
| | a-3 | P-17148, BP-6972 | 45 | 35 | ⊚ | ⊚ | ◯ | ◯˜Δ |
| | | P-17149, BP-6971 | 45 | 40 | ⊚ | ⊚˜◯ | ◯ | Δ |
| | | P-17150, BP-6973 | 55 | 50 | ⊚ | ⊚˜◯ | ◯˜Δ | Δ |
| Moderate | b-1 | P-17148, BP-6972 | 40 | 30 | ⊚ | ◯ | ◯˜Δ | Δ |
| nutrient | | P-17149, BP-6971 | 40 | 40 | ⊚ | ◯ | ◯˜Δ | Δ |
| medium | | P-17150, BP-6973 | 50 | 45 | ⊚ | ◯ | ◯˜Δ | Δ |
| | b-2 | P-17148, BP-6972 | 75 | 30 | ⊚ | ◯˜Δ | Δ˜X | Δ˜X |
| | | P-17149, BP-6971 | 30 | 35 | ⊚ | ◯˜Δ | Δ | Δ˜X |
| | | P-17150, BP-6973 | 40 | 45 | ⊚ | ◯˜Δ | Δ | X |
| | b-3 | P-17148, BP-6972 | 40 | 35 | ⊚ | ◯ | ◯˜Δ | Δ |
| | | P-17149, BP-6971 | 40 | 45 | ⊚ | ◯ | ◯˜Δ | Δ |
| | | P-17150, BP-6973 | 60 | 50 | ⊚ | ◯ | ◯˜Δ | Δ |
| High | c-1 | P-17148, BP-6972 | 35 | 30 | ◯ | ◯ | Δ | Δ˜X |
| nutrient | | P-17149, BP-6971 | 35 | 40 | ◯ | ◯˜Δ | Δ | Δ˜X |
| medium | | P-17150, BP-6973 | 50 | 50 | ⊚ | ◯ | Δ | Δ˜X |
| | c-2 | P-17148, BP-6972 | 30 | 30 | ◯ | Δ | X | X |
| | | P-17149, BP-6971 | 25 | 20 | ◯ | Δ | X | X |
| | | P-17150, BP-6973 | 30 | 35 | Δ | Δ˜X | X | X |
| | c-3 | P-17148, BP-6972 | 30 | 30 | ◯ | Δ˜X | X | X |
| | | P-17149, BP-6971 | 35 | 30 | ◯ | Δ | X | X |
| | | P-17150, BP-6973 | 45 | 45 | ◯ | Δ | Δ | X |

¹⁾odoriferous sulfur compounds;
²⁾odoriferous nitrogen compounds

TABLE 10
Lactobacillus clearans culture and overall correlation of
subcultures and titer

Number of subcultures and
extent of titer

| | 1^{st} | 3^{rd} | 6^{th} | 9^{th} |
|---|---|---|---|---|
| Low nutrient medium | ⊚ | ⊚˜∘ | ∘˜Δ | ∘˜Δ |
| Moderate nutrient medium | ⊚˜∘ | ∘˜Δ | ∘˜x | Δ˜x |
| High nutrient medium | ⊚˜Δ | ∘˜x | Δ˜x | Δ˜x |

In view of their characteristics, Lactobacillus clearans were subcultured with the addition of the enteric putrefying odoriferous substances comprising odoriferous sulfur, nitrogen, and carbon compounds added to low, moderate, and high nutrient culture media, the bacteria were transplanted to media for assaying the titer, and the titer was assayed, with the results given in Table 11. Here, F components are those including 0.2 g methyl sulfide, 0.3 g skatole, and 1 g butyric acid per liter medium. Other than F components, enteric putrefying odoriferous substances comprising odoriferous sulfur, nitrogen, and carbon compounds, such as sodium sulfide, mercaptane, indole, acetic acid, and propionic acid could be selected for the test without major differences in the results.

TABLE 11
Correlation between number of subcultures and titer of
Lactobacillus clearans in medium containing F components

| | | | | Decrease | | | | | |
|---|---|---|---|---|---|---|---|---|---|
| | | | | (%) by 72 | | | | | |
| | | | | hour in 1^{st} | | Number of subcultures | | | |
| | | Substance | Test bacterium | generation | | and titer | | | |
| Type | Medium | added | FERM No. | S¹⁾ | N²⁾ | 1^{st} | 3^{rd} | 6^{th} | 9^{th} |
| Low | a-1 | F | P-17148, BP-6972 | 50 | 40 | ⊚ | ⊚ | ⊚˜◯ | ◯ |
| nutrient | | compo- | P-17149, BP-6971 | 55 | 50 | ⊚ | ⊚ | ⊚˜◯ | ◯ |
| medium | | nents | P-17150, BP-6973 | 70 | 65 | ⊚ | ⊚ | ⊚˜◯ | ⊚˜◯ |
| | a-2 | F | P-17148, BP-6972 | 50 | 40 | ⊚ | ⊚ | ⊚˜◯ | ◯ |
| | | compo- | P-17149, BP-6971 | 50 | 75 | ⊚ | ⊚ | ⊚˜◯ | ◯ |
| | | nents | P-17150, BP-6973 | 65 | 60 | ⊚ | ⊚ | ⊚˜◯ | ◯ |
| | a-3 | F | P-17148, BP-6972 | 50 | 40 | ⊚ | ⊚ | ⊚˜◯ | ◯ |
| | | compo- | P-17149, BP-6971 | 50 | 50 | ⊚ | ⊚ | ⊚˜◯ | ◯˜Δ |
| | | nents | P-17150, BP-6973 | 65 | 60 | ⊚ | ⊚ | ⊚˜◯ | ◯˜Δ |
| Moderate | b-1 | F | P-17148, BP-6972 | 50 | 40 | ⊚ | ⊚ | ◯ | ◯˜Δ |
| nutrient | | compo- | P-17149, BP-6971 | 50 | 45 | ⊚ | ⊚ | ◯ | ◯˜Δ |
| medium | | nents | P-17150, BP-6973 | 60 | 60 | ⊚ | ⊚ | ◯ | ◯˜Δ |
| | b-2 | F | P-17148, BP-6972 | 50 | 40 | ⊚ | ◯ | ◯˜Δ | Δ |
| | | compo- | P-17149, BP-6971 | 55 | 50 | ⊚ | ⊚ | ◯ | ◯˜Δ |
| | | nents | P-17150, BP-6973 | 65 | 60 | ⊚ | ⊚ | ◯˜Δ | ◯ |
| | b-3 | F | P-17148, BP-6972 | 50 | 40 | ⊚ | ⊚ | ◯ | ◯ |
| | | compo- | P-17149, BP-6971 | 50 | 50 | ⊚ | ⊚ | ◯ | ◯˜Δ |
| | | nents | P-17150, BP-6973 | 65 | 60 | ⊚ | ⊚ | ⊚˜◯ | ◯ |
| High | c-1 | F | P-17148, BP-6972 | 50 | 40 | ◯ | ◯ | ◯˜Δ | Δ˜X |
| nutrient | | compo- | P-17149, BP-6971 | 50 | 45 | ◯ | ◯ | Δ | Δ˜X |
| medium | | nents | P-17150, BP-6973 | 60 | 60 | ⊚ | ◯ | ◯˜Δ | Δ |
| | c-2 | F | P-17148, BP-6972 | 45 | 35 | ⊚˜◯ | ◯ | Δ˜X | X |
| | | compo- | P-17149, BP-6971 | 45 | 45 | ⊚˜◯ | ◯ | Δ˜X | X |
| | | nents | P-17150, BP-6973 | 55 | 55 | ◯ | ◯ | Δ˜X | X |
| | c-3 | F | P-17148, BP-6972 | 45 | 35 | ⊚ | ◯ | Δ | X |
| | | compo- | P-17149, BP-6971 | 45 | 45 | ⊚ | ◯ | Δ | X |
| | | nents | P-17150, BP-6973 | 55 | 50 | ⊚˜◯ | ◯ | Δ | X |

¹⁾odoriferous sulfur compounds;
²⁾odoriferous nitrogen compounds

Table 11 shows that the addition of F components to the base medium as the subculture and growth media was important for preventing the Lactobacillus clearans titer from decreasing, this being particularly true when the base medium was high nutrient media. It was also clear that there was relatively less decrease in titer by the third generation of subculture when the F components were added. The titer decreased rapidly thereafter, however.

In terms of preservatives, Table 12 shows the changes in the viability of Lactobacillus clearans when protein-amino acid-based preservatives were added, Table 13 shows the changes in the viability of Lactobacillus clearans when saccharides and animal-based protein-vitamin-mineral complex preservatives were added, and Table 14 shows the changes in the viability of Lactobacillus clearans when plant protein-vitamin-mineral complex preservatives and other preservatives were added. Here, alfalfa juice and clover juice refer to liquids which are made by adding 10-fold water to alfalfa grass or clover grass to make a juice, these being used as alfalfa juice or clover juice, respectively. The amounts in which the preservatives are added are expressed in terms of the weight ratio of the preservative relative to the weight of the viable cells. For example, an amount of 10 indicates that preservative was added in an amount 10 times that of the viable cells. The viability of Lactobacillus clearans is expressed as a percentage, where 100% is the viable cell count immediately after lyophilization. The viability during lyophilization is indicated as concentric circles when the viability is more than 90%, as two concentric circles to one circle when the rate is 80 to 90%, as one circle when the rate is 60 to 80%, as a square when the rate is 40 to 60%, as a triangle when the rate is 20 to 40%, and as an “x” when the rate is less than 20%. Changes in the titer of Lactobacillus clearans during storage are indicated as ++ when the titer was maintained at a high level, as + when the titer decreased slightly, as ± when the titer clearly decreased, as − when the titer decreased considerably, and as −− when the titer decreased rapidly.

TABLE 12
Changes in viability of Lactobacillus clearans with use
of protein-amino acid-based preservatives

| | | Results | Changes over time during storage | | | |
|---|---|---|---|---|---|---|
| Preservative | | during | Viability (%) | | | Changes |
| | Amount | lyophil- | 3 | 6 | 12 | in |
| Type | added | ization | months | months | months | titer |
| Peptone | 10 | Δ | 30 | 10 | 2 | — |
| Casamino | 10 | □ | 50 | 35 | 20 | ± |
| acid | | | | | | |
| Cystine | 20 | ◯ | 70 | 50 | 35 | + |
| Cysteine | 20 | ◯ | 65 | 45 | 30 | + |
| Methionine | 20 | ◯ | 65 | 45 | 30 | + |
| Taurine | 20 | ◯ | 70 | 50 | 35 | + |
| Alanine | 20 | Δ | 30 | 15 | 5 | − |
| Glycine | 20 | Δ | 25 | 10 | 2 | − |
| Sodium | 20 | □ | 60 | 40 | 20 | ± |
| glutamate | | | | | | |
| Aminobu- | 20 | □ | 65 | 45 | 25 | ± |
| tyric acid | | | | | | |
| Leucine | 20 | Δ | 25 | 10 | 3 | − |
| Lysine | 20 | Δ | 30 | 15 | 5 | ± |
| Tryptophan | 20 | Δ | 25 | 10 | 4 | − |
| Arginine | 20 | Δ | 20 | 5 | 2 | − |
| Asparagine | 20 | □ | 55 | 30 | 15 | ± |
| Ovalbumin | 20 | ◯ | 70 | 50 | 30 | + |
| Soybean | 20 | ◯ | 60 | 45 | 25 | ± |
| whey | | | | | | |
| Yolk | 100 | ◯ | 50 | 40 | 30 | ± |
| Gelatin | 10 | X | 10 | 3 | 0 | — |

*expressed as weight ratio of preservative relative to viable cell weight

TABLE 13
Changes in viability of Lactobacillus clearans
with use of saccharides and animal-based
protein·vitamin·mineral complex preservatives

| | | Results | Changes over time during storage | | | |
|---|---|---|---|---|---|---|
| Preservative | | during | Viability (%) | | | Changes |
| | Amount | lyophil- | 3 | 6 | 12 | in |
| Type | added | ization | months | months | months | titer |
| Saccharides | | | | | | |
| Lactose | 100 | □ | 70 | 50 | 20 | ± |
| Soluble | 100 | □ | 60 | 40 | 15 | ± |
| starch | | | | | | |
| Potato | 100 | Δ | 30 | 20 | 10 | − |
| starch | | | | | | |
| Sucrose | 20 | Δ | 30 | 15 | 5 | − |
| Glucose | 20 | Δ | 25 | 15 | 5 | − |
| Trehalose | 40 | ◯ | 75 | 50 | 30 | + |
| Protein· | | | | | | |
| vitamin· | | | | | | |
| mineral | | | | | | |
| complexes | | | | | | |
| Skim | 150 | ◯ | 70 | 50 | 35 | ± |
| powdered | | | | | | |
| milk | | | | | | |
| Liver extract | 10 | □ | 60 | 40 | 20 | ± |
| Yeast extract | 10 | □ | 50 | 30 | 20 | ± |
| Heart extract | 10 | Δ | 25 | 15 | 5 | − |
| Equine | 250 | □ | 60 | 40 | 25 | ± |
| serum | | | | | | |
| Yeast (killed | | | | | | |
| Baker's | 100 | ◯ | 60 | 50 | 30 | + |
| yeast cells) | | | | | | |

*expressed as weight ratio of preservative relative to viable cell weight

TABLE 14
Changes in viability of Lactobacillus clearans with use
of plant protein·vitamin·mineral complexes and others

| | | Results | Changes over time during storage | | | |
|---|---|---|---|---|---|---|
| Preservative | | during | Viability (%) | | | Changes |
| | Amount | lyophil- | 3 | 6 | 12 | in |
| Type | added | ization | months | months | months | titer |
| Protein· | | | | | | |
| vitamin· | | | | | | |
| mineral | | | | | | |
| complexes | | | | | | |
| (plant) | | | | | | |
| Chlorella | 50 | ◯ | 65 | 45 | 35 | + |
| Chlorella | 20 | □ | 60 | 40 | 30 | ± |
| CGF | | | | | | |
| Rice bran | 20 | ◯ | 60 | 50 | 35 | + |
| Bran | 20 | ◯ | 55 | 45 | 33 | + |
| Alfalfa juice | 40 | □ | 50 | 35 | 25 | ± |
| (grass) | | | | | | |
| Powdered | 20 | Δ | 20 | 10 | 7 | − |
| miso | | | | | | |
| Azuki | 20 | Δ | 25 | 10 | 5 | − |
| powder | | | | | | |
| Soba noodle | 20 | X | 20 | 5 | 1 | — |
| flour | | | | | | |
| Clover juice | 250 | □ | 60 | 45 | 25 | ± |
| Wheat germ | 50 | □ | 65 | 5 | 25 | ± |
| extract | | | | | | |
| Soybean | 100 | ◯ | 70 | 55 | 35 | + |
| milk | | | | | | |
| Other | | | | | | |
| Tomato juice | 50 | □ | 50 | 35 | 20 | ± |
| Carrot juice | 50 | □ | 55 | 40 | 25 | + |
| Orange juice | 50 | X | 15 | 2 | 0.05 | — |
| Grape juice | 50 | □ | 50 | 30 | 20 | ± |
| Aloe powder | 10 | Δ | 35 | 20 | 10 | ± |
| Green tea | 20 | Δ | 35 | 20 | 15 | ± |
| powder | | | | | | |
| Physio- | 1.7 | X | 5 | 0 | 0 | — |
| logical | | | | | | |
| saline | | | | | | |

*expressed as weight ratio of preservative relative to viable cell weight

It is apparent from Tables 12, 13, and 14 that the use of preservatives affording high viability during the lyophilization of Lactobacillus clearans resulted in good viability during storage as well, and that lower decreases in the titer were obtained at the same time. That is, by just determining the viability during lyophilization, it was possible to make dramatic progress in subsequent research upon the rapid study of conditions, from the selection of the preservatives to the pre-freezing and lyophilization temperature, drying time, and the like, without taking very much time. As a result, it became clear that commonly used methods for conventionally known Lactobacillus were suitable for conditions such as the pre-freezing and lyophilization temperature and drying time during the handling of Lactobacillus clearans, but that the effectiveness of the preservative was the most important factor for sustaining the viability and titer.

Preservatives with no preservative effects when used on their own were excluded, and the remaining preservatives were studied in various combinations. Some of the results are given in Tables 15, 16, and 17. Here, the amounts in which the preservatives were added are expressed as the weight ratio of the preservative relative to the viable cell weight. For example, an amount of 10 indicates that preservative was added in an amount 10 times that of the viable cells. The viability of Lactobacillus clearans during lyophilization is indicated as two concentric circles when the viability is more than 90%, as two concentric circles to one circle when the rate is 80 to 90%, as one circle when the rate is 60 to 80%, as a square when the rate is 40 to 60%, as a triangle when the rate is 20 to 40%, and as an “x” when the rate is less than 20%. A comparison of Tables 13 and 14 reveals that the preservatives afforded better viability when used in combination than when used alone. The combined use of animal-derived preservatives with plant-derived types resulted in good viability during lyophilization, of course, but also in better effects during subsequent storage. The results are given in Table 18, where the changes in the titer of Lactobacillus clearans during storage are indicated as ++ when the titer was maintained at a high level, as + when the titer decreased slightly, as ± when the titer clearly decreased, as − when the titer decreased considerably, and as −− when the titer decreased rapidly.

TABLE 15
Changes in the viability of Lactobacillus clearans with
combinations of two types of preservatives

| Type of | | Results during |
|---|---|---|
| preservative | Amount added* | lyophilization |
| Ovalbumin | 10 | ◯˜⊚ |
| Trehalose | 20 | |
| Ovalbumin | 10 | ◯ |
| Skim milk | 100 | |
| Ovalbumin | 10 | ⊚ |
| Soybean milk | 70 | |
| Ovalbumin | 10 | ◯˜⊚ |
| Carrot juice | 35 | |
| Trehalose | 20 | ◯˜⊚ |
| Skim milk | 100 | |
| Trehalose | 20 | ⊚ |
| Soybean milk | 70 | |
| Trehalose | 20 | ◯ |
| Carrot juice | 35 | |
| Skim milk | 75 | ⊚ |
| Soybean milk | 50 | |
| Skim milk | 100 | ◯˜⊚ |
| Carrot juice | 35 | |
| Soybean milk | 70 | ◯ |
| Carrot juice | 35 | |

*expressed as weight ratio of preservative relative to viable cell weight

TABLE 16
Changes in the viability of Lactobacillus clearans with
combinations of three types of preservatives

| Type of | | Results during |
|---|---|---|
| preservative | Amount added* | lyophilization |
| Ovalbumin | 7 | ◯ |
| Trehalose | 15 | |
| Skim milk | 80 | |
| Ovalbumin | 7 | ◯˜⊚ |
| Trehalose | 15 | |
| Soybean milk | 65 | |
| Ovalbumin | 7 | ◯˜⊚ |
| Trehalose | 15 | |
| Carrot juice | 30 | |
| Ovalbumin | 7 | ⊚ |
| Skim milk | 70 | |
| Soybean milk | 45 | |
| Ovalbumin | 7 | ◯˜⊚ |
| Skim milk | 70 | |
| Carrot juice | 30 | |
| Trehalose | 15 | ⊚ |
| Skim milk | 70 | |
| Soybean milk | 45 | |
| Trehalose | 15 | ◯˜⊚ |
| Skim milk | 70 | |
| Carrot juice | 25 | |
| Skim milk | 15 | ⊚ |
| Soybean milk | 45 | |
| Carrot juice | 25 | |

*expressed as weight ratio of preservative relative to viable cell weight

TABLE 17
Changes in the viability of Lactobacillus clearans with
combinations of four to five types of preservatives

| Type of | Amount | Results during |
|---|---|---|
| preservative | added* | lyophilization |
| Ovalbumin | 7 | ⊚ |
| Trehalose | 15 | |
| Skim milk | 70 | |
| Soybean milk | 45 | |
| Ovalbumin | 7 | ∘˜⊚ |
| Trehalose | 15 | |
| Skim milk | 70 | |
| Carrot juice | 25 | |
| Ovalbumin | 7 | ⊚ |
| Trehalose | 15 | |
| Soybean milk | 45 | |
| Carrot juice | 25 | |
| Trehalose | 15 | ⊚ |
| Skim milk | 70 | |
| Soybean milk | 45 | |
| Carrot juice | 25 | |
| Ovalbumin | 7 | ⊚ |
| Trehalose | 15 | |
| Skim milk | 70 | |
| Soybean milk | 45 | |
| Carrot juice | 25 | |

*expressed as weight ratio of preservative relative to viable cell weight

TABLE 18
Changes in the viability and titer of Lactobacillus clearans with use
of preservatives affording more than 90% viability during lyophilization

| | Mean viablility over | | | |
|---|---|---|---|---|
| | time | | | |
| | 3 | 6 | 12 | Changes |
| | months | months | months | in titer |
| When using | 95% | 80% | 70% | ++ |
| preservatives | | | | |
| affording 90% | | | | |
| viability during | | | | |
| lyophilization | | | | |

It was apparent that excellent preservative effects were obtained with the joint use of substances with which lactobacilli adhere and live together in a symbiotic relationship in the natural world, such as rice bran, bran, yeast, chlorella, grasses such as clover, and fruits such as grapes, together with the substances rated with a single circle or square in Tables 12, 13, and 14, such as soybean milk.

It was also confirmed that the preservability could be further enhanced when substances effectively maintaining the titer, such as odoriferous sulfur, nitrogen, and carbon compounds, amino acids such as sulfur-containing amino acids and glutamic acid, vitamins such as Vitamin C and calcium pantothenate, minerals such as zinc and magnesium, and bile powder, were added, either by themselves or in combination, to highly effective preservatives during the subculture of Lactobacillus clearans.

Various forms of preservation were contemplated in addition to lyophilization as methods for preserving Lactobacillus clearans, such as wet preservation of liquids or cell masses, semi-dry preservation with about a 15% moisture content, dry preservation with a moisture content of about 8%, and ultracold preservation at between −40 and −196° C., but basically good results were obtained when preserved with preservatives that showed the excellent effects described above during use in lyophilization.

The present invention is described in detail below with reference to examples, but the scope of the present invention is not limited to these examples alone.

### Example 1

10 L medium (pH 7. 0) comprising 5 g meat extract (Wako Pure Chemical Industries, LTD. ), 5 g peptone (Wako Pure Chemical Industries, LTD.), 3 g sodium butyrate (Wako Pure Chemical Industries, LTD.), 1 mL aqueous ammonia (Wako Pure Chemical Industries, LTD.), 10 g glucose (Wako Pure Chemical Industries, LTD.), 0.5 g cystine (Wako Pure Chemical Industries, LTD.), and 2 g yeast extract (Nihon Seiyaku) per liter medium was inoculated with Lactobacillus clearans (FERM P-17150, BP-6973) for 72 hours of anaerobic culture at 37° C. The culture broth was centrifuged, giving 10 g of a cell mass. The mass was washed with 500 mL of physiological saline (prepared with sodium chloride from Wako Pure Chemical Industries, LTD.) and centrifuged twice. The resulting purified cell mass was introduced into a solution consisting of 500 mL soybean milk (by Tsujimoto Shokuhin Kogyo), 50 g skim milk (Snow Brand Milk Products, Co.), 30 g trehalose (Hayashibara KK), and 0.5 g cystine (Wako Pure Chemical Industries, LTD.) and thoroughly stirred. The mixture was lyophilized in vacuo by a common method to give 133 g bacterial cell preparation. The cell count was 3.0×10⁹ cells/g. The cell preparation was stored at room temperature with a silica gel dessicant (Manabe Kaseihin) and an oxygen absorbent (Mitsubishi Gas Chemical Co.), the viable cell count was studied over 18 months to calculate the viability, and the titer was assayed, with the results given in Table 19. Table 19 shows that a high Lactobacillus clearans titer was maintained, with virtually no drop.

TABLE 19
Example of changes in Lactobacillus clearans viable cell
count, viability, and titer

| Viable cell count/g | | | | | |
|---|---|---|---|---|---|
| Immediately | | | | | |
| after | 3 | 6 | 12 | 18 | Changes |
| preparation | months | months | months | months | in titer |
| 3.0 × 10⁹ | 2.8 × 10⁹ | 2.5 × 10⁹ | 2.0 × 10⁹ | 1.5 × 10⁹ | ++ |
| Viability | | 83 | 66 | 50 | |

### Example 2

10 L medium (pH 7.0) comprising 30 g soybean whey (Fuji Oil Co.), 1 g peptone (Wako Pure Chemical Industries, LTD.), 1 g cystine (Wako Pure Chemical Industries, LTD.), 3 g sodium acetate (Wako Pure Chemical Industries, LTD.), 0.2 g sodium sulfide (Wako Pure Chemical Industries, LTD.), 0.01 g calcium pantothenate (Wako Pure Chemical Industries, LTD.), and 2 g Baker's yeast (Oriental Yeast) per liter medium was inoculated with Lactobacillus clearans (FERM P-17149,BP-6971) for 72 hours of anaerobic culture at 37° C. The culture broth was centrifuged, giving 28 g of a cell mass consisting of Lactobacillus clearans and dead yeast cells. The mass was washed with 500 mL of physiological saline (prepared with sodium chloride from Wako Pure Chemical Industries, LTD.) and centrifuged twice. The resulting purified cell mass was introduced into 500 mL water containing 10 g dried chlorella (Yamaki), 25 g trehalose (Hayashibara KK), and 5 g soluble starch (Wako Pure Chemical Industries, LTD.) and thoroughly stirred The mixture was then lyophilized in vacuo by a common method to give 43 g bacterial cell preparation. The cell count was 10.0×10⁹ cells/g. The cell preparation was stored at room temperature in glass jars shielded from light along with a silica gel dessicant (Manabe Kaseihin) and an oxygen absorbent (Mitsubishi Gas Chemical Co.), the viable cell count was studied over 18 months to calculate the viability, and the titer was assayed, with the results given in Table 20. Table 20 shows that a high Lactobacillus clearans titer was maintained, with virtually no drop.

TABLE 20
Example of changes in Lactobacillus clearans viable cell
count, viability, and titer

| Viable cell count/g | | | | | |
|---|---|---|---|---|---|
| Immediately | | | | | |
| after | 3 | 6 | 12 | 18 | Changes |
| preparation | months | months | months | months | in titer |
| 10.0 × 10⁹ | 9.5 × 10⁹ | 8.5 × 10⁹ | 7.0 × 10⁹ | 5.0 × 10⁹ | ++ |
| Viability | | 85 | 70 | 50 | |

### Comparative Example 1

10 L medium (pH 7.0) comprising 10 g meat extract (Wako Pure Chemical Industries, LTD.), 10 g peptone (Wako Pure Chemical Industries, LTD.), 3 g yeast extract (Nihon Seiyaku), 10 g glucose (Wako Pure Chemical Industries, LTD.), 2 g K₂HPO₄, 1 g MgSO₄•7H₂O, 1 g NaCl, and 1 g CaCl•2H₂O per liter medium was inoculated with Lactobacillus clearans (FERM P-17150,BP-6973) for 72 hours of anaerobic culture at 37° C. The culture broth was centrifuged, giving 18 g of a cell mass. The mass was washed with 500 mL of physiological saline (prepared with sodium chloride from Wako Pure Chemical Industries, LTD.) and centrifuged twice. The resulting purified cell mass was introduced into 1000 mL of 20% soluble starch (Wako Pure Chemical Industries, LTD.) solution and thoroughly stirred. The mixture was lyophilized in vacuo by a common method to give 204 g bacterial cell preparation. The cell count was 4.5×10⁹ cells/g. The cell preparation was stored at room temperature with a silica gel dessicant (Manabe Kaseihin) and an oxygen absorbent (Mitsubishi Gas Chemical Co.), the viable cell count was studied over 18 months to calculate the viability, and the titer was assayed, with the results given in Table 21. Table 21 shows that not only did the viability decrease rapidly, but also that the titer decreased considerably, when Lactobacillus clearans was cultured in a common medium and preserved by a common method.

TABLE 21
Example of changes in Lactobacillus clearans viable cell
count, viability, and titer in common medium and preservation

| Viable cell count/g | | | | | |
|---|---|---|---|---|---|
| Immediately | | | | | |
| after | 3 | 6 | 12 | 18 | Changes |
| preparation | months | months | months | months | in titer |
| 4.5 × 10⁹ | 2.5 × 10⁹ | 1.5 × 10⁸ | 4.0 × 10⁹ | 0 | −− |
| Viability | 55 | 33 | 9 | 0 | |

### Comparative Example 2

10 L medium (pH 7.0) comprising 10 g meat extract (Wako Pure Chemical Industries, LTD.), 10 g peptone (Wako Pure Chemical Industries, LTD.), 3 g yeast extract (Nihon Seiyaku), 10 g glucose (Wako Pure Chemical Industries, LTD.), 2 g K₂HPO₄, 1 g MgSO₄•7H₂O, 1 g NaCl, and 1 g CaCl₂•2H₂O per liter medium was inoculated with Lactobacillus clearans (FERM P-17150,BP-6973) for 72 hours of anaerobic culture at 37° C. The culture broth was centrifuged, giving 18 g of a cell mass. The mass was washed with 500 mL of physiological saline (prepared with sodium chloride from Wako Pure Chemical Industries, LTD.) and centrifuged twice. The resulting purified cell mass was introduced into a solution consisting of 900 mL soybean milk (by Tsujimoto Shokuhin Kogyo), 90 g skim milk (Snow Brand Milk Products, Co.), 54 g trehalose (Hayashibara KK), and 0.9 g cystine (neutral) (Wako Pure Chemical Industries, LTD.) and thoroughly stirred. The mixture was lyophilized in vacuo by a common method to give 239 g bacterial cell preparation. The cell count was 5.0×10⁹ cells/g. The cell preparation was stored at room temperature with a silica gel dessicant (Manabe Kaseihin) and an oxygen absorbent (Mitsubishi Gas Chemical Co.), the viable cell count was studied over 18 months to calculate the viability, and the titer was assayed, with the results given in Table 22. Table 22 shows that good viability was obtained, although not as good as in Example 1, when Lactobacillus clearans was cultured by a common method and preserved by the method of the present invention. The titer, however, decreased somewhat during culture, and thereafter tended to continue decreasing, albeit gradually.

TABLE 22
Example of changes in Lactobacillus clearans viable cell
count, viability, and titer in common medium but with
preservation by the present invention

| Viable cell count/g | | | | | |
|---|---|---|---|---|---|
| Immediately | | | | | |
| after | 3 | 6 | 12 | 18 | Changes |
| preparation | months | months | months | months | in titer |
| 5.0 × 10⁹ | 4.5 × 10⁹ | 3.8 × 10⁹ | 2.5 × 10⁹ | 2.0 × 10⁹ | ± |
| Viability | 90 | 76 | 50 | 40 | |

### Comparative Example 3

10 L medium (pH 7.0) comprising 5 g meat extract (Wako Pure Chemical Industries, LTD.), 5 g peptone (Wako Pure Chemical Industries, LTD.), 3 g sodium butyrate (Wako Pure Chemical Industries, LTD.), 1 g aqueous ammonia (Wako Pure Chemical Industries, LTD.), 10 g glucose (Wako Pure Chemical Industries, LTD.), 0.5 g cystine (Wako Pure Chemical Industries, LTD.), and 2 g yeast extract (Nihon Selyaku) per liter medium was inoculated with Lactobacillus clearans (FERM P-17150,BP-6973) for 72 hours of anaerobic culture at 37° C. The culture broth was centrifuged, giving 10 g of a cell mass. The mass was then washed with 500 mL of physiological saline (prepared with sodium chloride from Wako Pure Chemical Industries, LTD.) and centrifuged twice. The resulting purified cell mass was introduced into 550 mL of 20% soluble starch (Wako Pure Chemical Industries, LTD.) solution and thoroughly stirred. The mixture was lyophilized in vacuo by a common method to give 112 g bacterial cell preparation. The cell count was 2.5×10⁹ cells/g. The cell preparation was stored at room temperature with a silica gel dessicant (Manabe Kaseihin) and an oxygen absorbent (Mitsubishi Gas Chemical Co.), the viable cell count was studied over 18 months to calculate the viability, and the titer was assayed, with the results given in Table 23. Table 23 shows that the viability tended to be the same as that in Comparative Example 1 when Lactobacillus clearans was cultured in the medium of the present invention and preserved by a common method. The titer did not drop as rapidly as it did in Comparative Example 1, but did tend to decrease gradually.

TABLE 23
Example of changes in Lactobacillus clearans viable cell
count, viability, and titer in medium of present
invention but by common preservation

| Viable cell count/g | | | | | |
|---|---|---|---|---|---|
| Immediately | | | | | |
| after | 3 | 6 | 12 | 18 | Changes |
| preparation | months | months | months | months | in titer |
| 2.5 × 10⁹ | 2.5 × 10⁹ | 1.0 × 10⁹ | 3.8 × 10⁸ | 1.0 × 10⁸ | − |
| Viability | 60 | 40 | 15 | 0.4 | |

Lactobacillus clearans are novel strains of lactobacilli that utilize and degrade odoriferous sulfur, nitrogen, and carbon compounds, and have been noted as effective strains demonstrating a potent purifying capacity in the intestines because of the above functions. However, there are no known methods such as this for obtaining lactobacilli with a high titer exhibiting such functions, or methods for sustaining the high titer that is obtained for long periods of time.

The use of the culture medium in the present invention allows Lactobacillus clearans with a high titer to be cultured in a stable manner, so that odoriferous noxious sulfur, nitrogen, and carbon compounds can be readily degraded and eliminated. Accordingly, not only are enteric odoriferous noxious substances decreased, but bacteria regarded as being beneficial to the intestinal flora can be dramatically increased, while the growth of deleterious bacteria can be strongly suppressed.

The use of the method of preservation in the present invention allows Lactobacillus clearans with a high titer to be preserved for long periods of time, so that odoriferous noxious sulfur, nitrogen, and carbon compounds can be readily degraded and eliminated whenever required.

What is claimed is:

1. A culture medium for culturing Lactobacillus clearans, comprising the addition of at least one or both of sodium sulfide and aqueous ammonia, so that, during the culture of the Lactobacillus clearans, at least one or both of sodium sulfide and ammonia is decreased by Lactobacillus clearans by the 24th hour of culture.

2. A culture medium for culturing Lactobacillus clearans, comprising the addition of at least one or more of odoriferous sulfur compounds, odoriferous nitrogen compounds, and odoriferous carbon compounds to the culture medium as set forth in claim 1.

3. A culture medium for culturing Lactobacillus clearans, wherein the odoriferous sulfur compound as set forth in claim 2 is at least one or more of sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, and derivatives thereof.

4. A culture medium for culturing Lactobacillus clearans, wherein the odoriferous nitrogen compound as set forth in claim 2 is at least one or more of ammonia, skatole, indole, acetanilide, methylamine, dimethylamine, diethylamine, triethylamine, and derivatives thereof.

5. A culture medium for culturing Lactobacillus clearans, wherein the odoriferous carbon compound as set forth in claim 2 is at least one or more of formic acid, acetic acid, propionic acid, butyric acid, formaldehyde, acetaldehyde, propionaldehyde, crotonaldehyde, phenol, butyl alcohol, amyl alcohol, and derivatives thereof.

6. A culture medium for culturing Lactobacillus clearans, comprising the addition of at least one or more of sulfur-containing amino acid, glutamic acid, lysine, and aspartic acid as an amino acid to a culture medium as set forth in any of claims 1 through 5.

7. A culture medium for culturing Lactobacillus clearans, comprising the addition of at least one or more of Vitamin C, Vitamin E, Vitamin B₁₂, calcium pantothenate, folic acid, and nicotinamide as a vitamin to a culture medium as set forth in any of claims 1 through 6.

8. A culture medium for culturing Lactobacillus clearans, comprising the addition of at least one or more of manganese, zinc, magnesium, and molybdenum as a mineral component to a culture medium as set forth in any of claims 1 through 7.

9. A culture medium for culturing Lactobacillus clearans, comprising the addition of at least one or more of chlorella CGF, soybean milk, and bile powder to a culture medium as set forth in any of claims 1 through 8.

10. A culture medium for culturing Lactobacillus clearans, comprising the addition of sodium sulfide in a concentration of 500 ppm to a culture medium as set forth in any of claims 1 through 9, wherein the sodium sulfide is decreased 10% or more by the 24th hour of culture during the culture of Lactobacillus clearans.

11. A culture medium for culturing Lactobacillus clearans, comprising the addition of aqueous ammonia in a concentration of 500 ppm to a culture medium as set forth in any of claims 1 through 9, wherein the ammonia is decreased 10% or more by the 24th hour of culture during the culture of Lactobacillus clearans.

12. A method for preserving Lactobacillus clearans, comprising the presence of at least one or more of sulfur-containing amino acid, ovalbumin, bile powder, trehalose, raffinose, dead yeast cells, chlorella, rice bran, bran, soybean milk, and carrot juice as a preservative around the Lactobacillus clearans during the preservation of Lactobacillus clearans.

13. A method for preserving Lactobacillus clearans, comprising, in addition to a preservative as set forth in claim 12, the addition of at least one or more of glutamic acid, lysine, aspartic acid, Vitamin C, Vitamin E, Vitamin B₁₂, calcium pantothenate, folic acid, nicotinamide, manganese, zinc, magnesium, molybdenum, sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, ammonia, skatole, indole, acetanilide, methylamine, dimethylamine, diethylamine, triethylamine, formic acid, acetic acid, propionic acid, butyric acid, formaldehyde, acetaldehyde, propionaldehyde, crotonaldehyde, phenol, butyl alcohol, amyl alcohol, and derivatives thereof around the Lactobacillus clearans.

14. A method for preserving Lactobacillus clearans, comprising the presence of at least one or more of animal-derived powdered skim milk, ovalbumin, lactose, liver extract powder, and serum, as well as at least one or more of vegetable-derived soybean whey, trehalose, raffinose, starch, chlorella, chlorella CGF, rice bran, bran, alfalfa juice, clover juice, wheat germ extract, soybean milk, tomato juice, carrot juice, grape juice, aloe powder, green tea powder, yeast extract powder, and dead yeast cells as a preservative around the Lactobacillus clearans during the preservation of Lactobacillus clearans.

15. A method for preserving Lactobacillus clearans, comprising, in addition to a preservative as set forth in claim 14, the addition of at least one or more of glutamic acid, lysine, aspartic acid, Vitamin C, Vitamin E, Vitamin B₁₂, calcium pantothenate, folic acid, nicotinamide, manganese, zinc, magnesium, molybdenum, sodium sulfide, hydrogen sulfide, ammonium sulfide, methyl mercaptane, ethyl mercaptane, dimethyl mercaptane, dimethyl sulfide, dimethyl disulfide, diethyl sulfide, dibutyl sulfide, ammonia, skatole, indole, acetanilide, methylamine, dimethylamine, diethylamine, triethylamine, formic acid, acetic acid, propionic acid, butyric acid, formaldehyde, acetaldehyde, propionaldehyde, crotonaldehyde, phenol, butyl alcohol, amyl alcohol, and derivatives thereof around the Lactobacillus clearans.

16. A preservative method for preserving Lactobacillus clearans as set forth in any of claims 12 through 15, wherein the preservative method comprises any of lyophilization, ultracold preservation, or liquid, wet, semi-dry, or dry methods.
